(19) United States
(12) Patent Application Publication
(10) Pub. No.: US 2023/0141965 A1
(43) Pub. Date: May 11, 2023

(54) TREATMENT OF INFECTIOUS DISEASES

(71) Applicants: Durect Corporation, Cupertino, CA (US); Virginia Commonwealth University, Richmond, VA (US)

(72) Inventors: SHUNLIN REN, Richmond, VA (US); WEIQI LIN, Emerald Hills, CA (US); JAMES E. BROWN, Los Gatos, CA (US)

(21) Appl. No.: 17/794,804

(22) PCT Filed: Feb. 10, 2021
(86) PCT No.: PCT/US21/17467

§ 371 Date: Jul. 22, 2022

Related U.S. Application Data
(60) Provisional application No. 62/975,140, filed on Feb. 11, 2020.
(60) Provisional application No. 62/975,632, filed on Feb. 12, 2020.
(60) Provisional application No. 63/003,144, filed on Mar. 31, 2020.
(60) Provisional application No. 63/022,856, filed on May 11, 2020.
(60) Provisional application No. 63/044,264, filed on Jun. 25, 2020.

(51) Int. Cl. A61K 31/675; A61K 31/575; A61P 31/14

(52) U.S. Cl. CPC A61K 31/675; A61K 31/575; A61P 31/14

(57) ABSTRACT

Methods of treating infectious diseases are provided. For instance, the methods may comprise administering at least one antimicrobial in combination with at least one oxygenated cholesterol sulfate (OCS). Methods for preventing and/or treating organ dysfunction and/or organ failure for patients with an infectious disease are described herein. Methods for preventing and/or treating tissue damage of one or more organs or organ systems for patients with an infectious disease are described herein.

### BACKGROUND

Infectious diseases are caused by pathogenic microorganisms, such as bacteria, viruses, parasites or fungi. Infectious diseases can be spread, directly or indirectly, from one person to another.

For instance, human coronaviruses, first identified in the mid-1960s, are common viruses that infect most people at some time in their life, generally causing mild to moderate upper respiratory and gastrointestinal tract illnesses. The novel coronavirus referred to as “Middle East Respiratory Syndrome Coronavirus” (MERS-CoV or MERS) was first reported in Saudi Arabia in 2012 and has spread to several other countries. SARS-CoV, the coronavirus responsible for Severe Acute Respiratory Syndrome (SARS), was first recognized in China in 2002 and led to a worldwide outbreak in 2002 and 2003.

2019-nCoV acute respiratory disease, designated as novel coronavirus pneumonia (NCP) by China, and designated as the coronavirus disease 2019 (COVID-19) by the WHO, is an infectious respiratory disease caused by Severe Acute Respiratory Syndrome coronavirus 2 (SARS-CoV-2) (previously known as the 2019 novel coronavirus (2019-nCoV)), first detected during the 2019-2020 Wuhan coronavirus outbreak.

Patients with infectious diseases, such as COVID-19, are often at higher risk of becoming severely ill if they have underlying illness such as cardiovascular disease, liver disease, and kidney disease. Further, infectious diseases, such as COVID-19, are not only capable of causing disease associated with the respiratory system (e.g., infection of the upper respiratory tract, pneumonia, etc.), but may also cause damage to other organs such as the heart, the liver, and the kidneys.

U.S. Published Application No. 20170071964, which is incorporated by reference herein, discloses methods for treating Arenaviridae and Coronaviridae virus infections by administering nucleosides and prodrugs thereof.

U.S. Pat. No. 8,399,441, which is incorporated by reference herein, discloses the use of 5-cholesten-3,25-diol, 3-sulfate (25HC3S) and salts thereof for the treatment of conditions associated with high cholesterol and/or high triglycerides and/or inflammation (e.g., hypercholesterolemia, hypertriglyceridemia, non-alcoholic fatty liver diseases, atherosclerosis, etc.).

U.S. Pat. No. 9,034,859, which is incorporated by reference herein, discloses the use of 25HC3S and salts thereof for prevention and treatment of liver damage or disease.

U.S. Pat. No. 10,272,097, which is incorporated by reference herein, discloses oxygenated cholesterol sulfates, e.g., 25HC3S and salts thereof, for preventing and/or treating ischemia, organ dysfunction and/or organ failure, including multiple organ dysfunction syndrome (MODS), and necrosis and apoptosis associated with organ dysfunction/failure.

ClinicalTrials.gov includes disclosure of a research study to assess the safety, pharmacokinetics and pharmacodynamics of 25HC3S in patients with alcoholic hepatitis (AH), with dose escalation including three doses: 30 mg, 90 mg, and 150 mg. See ClinicalTrials.gov Identifier: NCT03432260.

There is an urgent need for improved methods to treat infectious diseases. In addition, there is an urgent need for improved methods to prevent and/or treat organ dysfunction and/or organ failure for patients with an infectious disease.

### SUMMARY

Provided are methods and compositions for the treatment of infectious diseases.

In some cases, the present disclosure relates to methods for treating an infectious disease in a human in need thereof comprising administering a therapeutically effective amount of at least one antimicrobial and at least one oxygenated cholesterol sulfate (OCS).

In some cases, the present disclosure relates to treatment of infections caused by the Ebolavirus, Arenaviridae and/or Coronaviridae virus families.

In some cases, the present disclosure includes a variety of methods of treating disease caused by a coronavirus infection, such as COVID-19, which is caused by Severe Acute Respiratory Syndrome coronavirus 2 (SARS-CoV-2) (previously known as novel coronavirus (2019-nCoV)). In certain cases, methods include treating a subject with COVID-19 caused by SARS-CoV-2 coronavirus infection where the virus has one or more mutations. In some cases, the subject is infected with SARS-CoV-2 coronavirus having one or more mutations selected from K417N, L452R, E484K, N501K, N501Y, D614G and P681H. In some cases, methods include treating a subject with COVID-19 that is caused by infection by one or more of SARS-CoV-2 coronavirus of lineage B.1.1.207 (having a P681H mutation), SARS-CoV-2 coronavirus of lineage B.1.1.7 (having a N501Y mutation), SARS-CoV-2 coronavirus Cluster 5, SARS-CoV-2 coronavirus variant 501.V2 (having N501Y, K417N and E484K mutations), SARS-CoV-2 coronavirus of lineage P.1 (having N501Y and E484K mutations) and SARS-CoV-2 coronavirus of lineage B.1.429 (having L452R mutation).

In some aspects, the present disclosure relates generally to methods and compounds for treating disease caused by Lassa virus infection and disease caused by Junin virus infection. The present disclosure relates generally to methods and compositions for treating Coronaviridae virus infections, such as methods involving at least one antiviral and at least one OCS for treating disease caused by SARS virus infection and MERS virus infection.

In some embodiments, the method comprises treating a Coronaviridae infection in a human in need thereof by administering a therapeutically effective amount of at least one antiviral and at least one OCS.

In some embodiments, the method comprises treating a MERS virus infection in a human in need thereof by administering a therapeutically effective amount of at least one antiviral and at least one OCS.

In some embodiments, the method comprises treating a SARS virus infection in a human in need thereof by administering a therapeutically effective amount of at least one antiviral and at least one OCS.

In some embodiments, the method of treating an infectious disease in a human in need thereof comprises administering at least one antimicrobial and at least one OCS, in combination with a pharmaceutically acceptable diluent or carrier.

In some embodiments, the method of treating an infectious disease in a human in need thereof comprises administering a therapeutically effective amount of a pharmaceutical composition comprising at least one antimicrobial and at least one OCS, in combination with at least one additional therapeutic agent.

Also provided are methods of preventing or treating dysfunction or failure of one or more organs or organ systems in a subject in need thereof, comprising: (1) identifying a subject with an infectious disease; and (2) administering to the subject an amount of at least one oxygenated cholesterol sulfate (OCS) that is sufficient to prevent or treat the dysfunction or failure of the organ or organ system. Aspects of the present disclosure also include methods of preventing or treating tissue damage of an organ or organ system in a subject in need thereof, comprising: (1) identifying a subject with an infectious disease; and (2) administering to the subject an amount of at least one oxygenated cholesterol sulfate (OCS) that is sufficient to prevent or treat tissue damage of the organ or organ system.

In some embodiments, methods of the present disclosure include (1) identifying a subject with an infectious disease; (2) identifying whether the subject has dysfunction or failure of an organ or organ system prior to treating the subject; and (3) administering to the subject an amount of at least one oxygenated cholesterol sulfate (OCS) that is sufficient to prevent or treat the dysfunction or failure of the organ or organ system. In some embodiments, methods of the present disclosure include (1) identifying a subject with an infectious disease; (2) identifying that the subject has dysfunction or failure of an organ or organ system caused by the infectious disease; and (3) administering to the subject an amount of at least one oxygenated cholesterol sulfate (OCS) that is sufficient to treat the dysfunction or failure of the organ or organ system caused by the infectious disease.

In some embodiments, methods of the present disclosure include (1) identifying a subject with an infectious disease; (2) identifying whether the subject has tissue damage of an organ or organ system prior to treating the subject; and (3) administering to the subject an amount of at least one oxygenated cholesterol sulfate (OCS) that is sufficient to prevent or reduce or treat the tissue damage of the organ or organ system. In some embodiments, methods of the present disclosure include (1) identifying a subject with an infectious disease; (2) identifying that the subject has tissue damage of an organ or organ system caused by the infectious disease; and (3) administering to the subject an amount of at least one oxygenated cholesterol sulfate (OCS) that is sufficient to treat the tissue damage of the organ or organ system caused by the infectious disease.

In some embodiments, methods include preventing or treating dysfunction or failure of one or more organs or organ systems in a human subject in need thereof and suffering from an infectious disease, comprising administering to the subject an amount of at least one oxygenated cholesterol sulfate (OCS) that is sufficient to prevent or treat the dysfunction or failure of the organ or organ system.

In some embodiments, methods include preventing or treating tissue damage of an organ or organ system in a human subject in need thereof and suffering from an infectious disease, comprising administering to the subject an amount of at least one oxygenated cholesterol sulfate (OCS) that is sufficient to prevent or treat the damage of the organ or organ system.

In some embodiments, methods include treating an infectious disease in a human subject in need thereof, the method comprising administering to the subject at least one oxygenated cholesterol sulfate (OCS) in an amount that is sufficient to treat the infectious disease.

In some embodiments, the one or more organs comprise at least one member selected from the lungs, the liver, the kidney, the heart, the brain, and the pancreas.

In some embodiments, the dysfunction or failure of one or more organs or organ systems is Multiple Organ Dysfunction Syndrome (MODS).

In some embodiments, the subject had liver disease prior to being infected with the infectious disease. The liver disease may comprise acute liver disease. The liver disease may also comprise chronic liver disease.

In some embodiments, the subject had kidney disease prior to being infected with the infectious disease. The kidney disease may comprise acute kidney injury. The kidney disease may comprise chronic kidney disease.

In some embodiments, the subject had lung disease prior to being infected with the infectious disease.

### BRIEF DESCRIPTION OF THE DRAWINGS

FIG. 1 shows that 25HC3S protected mice from lipopolysaccharide (LPS). FIG. 1 shows that treatment with 25HCS improved kidney, lung, and liver tissues.

FIG. 2 shows that 25HC3S protected mice from acetaminophen. FIG. 2 shows that treatment with 25HC3S improved kidney, lung, and liver tissues.

### DETAILED DESCRIPTION OF THE INVENTION

Methods for treating infectious diseases are described herein. The methods include administering to a human patient an amount of at least one antimicrobial and at least one oxygenated cholesterol sulfate (OCS) that is effective or sufficient to treat the infectious disease.

Methods for preventing and/or treating organ dysfunction and/or organ failure for patients with an infectious disease are described herein.

Methods for preventing and/or treating tissue damage of one or more organs or organ systems for patients with an infectious disease are described herein.

Patient outcomes are improved by mitigating severe downstream impacts associated with the infectious disease, such as sepsis and/or organ damage, dysfunction and/or failure, while other therapies and/or the patient's own immune system fight the infectious agent (e.g., viral infection) itself

### DEFINITIONS

The following definitions are used throughout:

“Treat” (treatment, treating, etc.) as used herein refers to administering at least one oxygenated cholesterol sulfate (OCS) and optionally at least one antimicrobial to a human subject that: (1) already exhibits at least one symptom of infectious disease; and/or (2) is diagnosed as having infectious disease, such as by a trained clinical professional; and/or (3) is determined to have infectious disease based on laboratory (e.g., molecular indicators) or clinical tests of one or more body fluids, such as mucus, saliva, blood. In other words, at least one parameter that is known to be associated with infectious disease has been measured, detected or observed in the subject. “Treatment” of infectious disease involves the lessening or attenuation, or in some instances, the complete eradication, of at least one symptom of infectious disease that was present prior to or at the time of administration of the at least one oxygenated cholesterol sulfate (OCS) and optionally the at least one antimicrobial. In some embodiments, treating infectious disease according to the present disclosure is sufficient to improve laboratory or clinical indicators of infectious disease in the subject as described in greater detail below. In certain instances, the improvement in the laboratory or clinical indicators of infectious disease in the subject is such that the subject is considered to no longer have infectious disease.

As used herein, “prophylactically treat” (“prophylactic treatment,” “prophylactically treating,” etc.) and “prevent” (“prevention,” preventing,” etc.) refer to warding off or averting the occurrence of at least one symptom of a disease or unwanted condition such as tissue damage or organ dysfunction or failure, by prophylactic administration of at least one OCS and optionally at least one antimicrobial to a subject in need thereof. Generally, “prophylactic” or “prophylaxis” relates to a reduction in the likelihood of the patient developing a condition or disorder. Typically, the subject is considered by one of skill in the art to be at risk of or susceptible to developing at least one symptom of the disease or unwanted condition, or is considered to be likely to develop at least one symptom of the disease/condition in the absence of medical intervention. Generally, however, for “prevention” or “prophylactic treatment,” administration occurs before the subject has, or is known or confirmed to have, symptoms of the disease (condition, disorder, syndrome, etc.; unless otherwise indicated, these terms are used interchangeably herein). In other words, symptoms may not yet be overt or observable. The subject may be considered at risk due to a variety of factors, including but not limited to: genetic predisposition; a pre-existing condition; possible or confirmed infection; recent certain or suspected or unavoidable or likely future exposure to a toxic agent (e.g., a toxic chemical or medication, radiation, infectious agent, etc.); or exposure to or experience of another stressor or combination of stressors that is/are linked to or associated with the development of the disease/condition which is being prevented. In some aspects of the prevention of tissue damage or organ dysfunction/failure, the subject may already display symptoms of a potential precursor of tissue damage or organ dysfunction/failure, for example, ischemia, sepsis, a harmful or inappropriate level of inflammation, deleterious cell death, necrosis, etc. In such aspects, treatment of the subject may prevent the noxious or harmful effects or outcomes (results) of the precursor condition. “Prevention” or “prophylactic treatment” of a disease or condition may involve completely preventing the occurrence of detectable symptoms, or, alternatively, may involve lessening or attenuating the degree, severity or duration of at least one symptom of the disease that would occur in the absence of the medical interventions provided herein, i.e., unless one or more OCSs and optionally at least one antimicrobial are administered. Alternatively, the subject may be experiencing early stage symptoms and what is prevented is the progression to full-blown disease.

As used herein, “organ” refers to a differentiated and/or relatively independent body structure comprising cells and tissues that performs some specialized function in the body of an organism. An “organ system” refers to two or more organs that work together in the execution of a body function. A hollow organ is an internal visceral organ (viscus) that forms a hollow tube or pouch, or that includes a cavity. Exemplary organs, the dysfunction or failure of which are prevented and/or treated by the administration of or contact with one or more OCS and optionally at least one antimicrobial, include but are not limited to: heart, lungs, (e.g., lungs damaged by pulmonary fibrosis, e.g., associated with chronic asthma), liver, pancreas, kidneys, brain, intestines, colon, thyroid, etc. In some cases, the dysfunction or failure which is prevented and/or treated by the administration of the one or more OCS and optionally at least one antimicrobial involves an organ other than the liver, for example heart, lungs, pancreas, kidneys, brain, intestines, colon, etc. In general, methods and compositions described herein that refer to “organs” should also be understood to include “organ systems”, unless otherwise specified.

“Organ dysfunction” denotes a condition or a state of health where an organ does not perform its expected function. Organ function represents the expected function of the respective organ within physiologic ranges. The person skilled in the art is aware of the respective function of an organ during medical examination. Organ dysfunction typically involves a clinical syndrome in which the development of progressive and potentially reversible physiological dysfunction in an organ, optionally in the absence of anatomic injuries.

“Organ failure” denotes an organ dysfunction to such a degree that normal homeostasis cannot be maintained without external clinical intervention.

“Acute organ dysfunction” refers to reduced organ function that occurs rapidly—in days or weeks (e.g., within 26 weeks, within 13 weeks, within 10 weeks, within 5 weeks, within 4 weeks, within 3 weeks, within 2 weeks, within 1 week, within 5 days, within 4 days, within 3 days, or within 2 days)—usually in a person who has no pre-existing disease.

“Acute organ failure” refers to loss of organ function that occurs rapidly—in days or weeks (e.g., within 26 weeks, within 13 weeks, within 10 weeks, within 5 weeks, within 4 weeks, within 3 weeks, within 2 weeks, within 1 week, within 5 days, within 4 days, within 3 days, or within 2 days)—usually in a person who has no pre-existing disease. For instance, the term “acute renal failure” means a rapid deterioration in renal function sufficient to result in accumulation of waste products in the body. Acute liver failure is discussed in more detail below,

“Pharmaceutically acceptable” refers to a substance that does not interfere with the effectiveness of the biological activity of the active ingredient and is not toxic to the host to which it is administered.

### Patient Population

The patient to be treated has an infectious disease caused by a pathogenic microorganism, such as a bacteria, virus, parasite or fungus.

For instance, methods of treatment herein include those for treating coronavirus infections in a human, including infections caused by alpha coronaviruses 229E (HCoV-229E) and NL63 (HCoV-NL63, New Haven coronavirus), beta coronaviruses OC43 (HCoV-OC43), HKU1, SARS-CoV (the coronavirus responsible for Severe Acute Respiratory Syndrome, or SARS), MERS-CoV (the coronavirus responsible for Middle East Respiratory Syndrome), previously known as Novel coronavirus 2012 and HCoV-EMC, and Severe Acute Respiratory Syndrome coronavirus 2 (SARS-CoV-2) (previously known as novel coronavirus (2019-nCoV)). In certain cases, methods include treatment of subjects infected by SARS-CoV-2 coronavirus that has one or more mutations. In some cases, the subject is infected with SARS-CoV-2 coronavirus having one or more mutations selected from K417N, L452R, E484K, N501K, N501Y, D614G and P681H. In some cases, methods include treating a subject with COVID-19 that is caused by infection by one or more of SARS-CoV-2 coronavirus of lineage B.1.1.207 (having a P681H mutation), SARS-CoV-2 coronavirus of lineage B.1.1.7 (having a N501Y mutation), SARS-CoV-2 coronavirus Cluster 5, SARS-CoV-2 coronavirus variant 501.V2 (having N501Y, K417N and E484K mutations), SARS-CoV-2 coronavirus of lineage P.1 (having N501Y and E484K mutations) and SARS-CoV-2 coronavirus of lineage B.1.429 (having L452R mutation). For the avoidance of doubt, terms herein such as “infection caused by SARS-CoV-2”, “coronavirus infection caused by SARS-CoV-2”, and “COVID-19” are used interchangeably.

In embodiments, the patient to be treated may be a human subject including newborns, infants, children and adults. In some embodiments, the patient is a human subject that is aged 17 years or younger, such as 15 years or younger, such as 10 years or younger, such as 9 years or younger, such as 8 years or younger, such as 7 years or younger, such as 6 years or younger, such as 5 years or younger, such as 4 years or younger, such as 3 years or younger, such as 2 years or younger, such as 1 year or younger, such as 6 months or younger, such as 1 month or younger and including a newborn human subject. In some embodiments, the patient is a human subject that is from age 18 to 44 years, such as from age 20 to 40 years, such as from age 25 to 35 years. In some embodiments, the patient is a human subject that is from age 45 to 65 years, such as from age 50 to 60 years. In certain embodiments, the patient is a human subject that is age 65 years or older, such as age 70 years or older, such as age 75 years or older, such as age 80 years or older, such as age 85 years or older, such as age 90 years or older, such as age 95 years or older and including a human subject that is age 100 years or older.

In embodiments, the subject may have a body mass index (BMI) of greater than or equal to 25 (e.g. greater than or equal to 30), where the BMI is calculated as 703 multiplied the body mass in lbs, then divided by the square of the body height in inches.

It has been found that COVID-19 in some cases disproportionately affects subjects of different ethnicities. The subject may self-identify as an ethnicity which is Black, African, Caribbean, Asian (including Indian, Pakistani, Bangladeshi and Chinese), Arab, American Indian or a mixed or multiple ethnic group.

In some cases, the patient to be treated according to the subject methods and compositions has one or more pre-existing conditions prior to being infected by the infectious disease. For instance, patients treated may have or have been diagnosed as having organ or organ system dysfunction and/or failure, such as dysfunction or failure of one or more components of the cardiovascular system, respiratory system, renal system, haematological system, neurological system, gastrointestinal organs, hepatic organs, heart, liver, lungs, intestines, colon, kidneys, spleen, or brain.

In some embodiments, the patient to be treated may continue to have the pre- existing condition when treated for the infectious disease, such as where the pre-existing condition (e.g., organ or organ system dysfunction/failure) is co-morbid with the infectious disease. In some embodiments, the pre-existing condition remains co-morbid with the infectious disease through the duration of treatment. In other embodiments, the pre-existing condition ceases prior to the end of illness caused by the infectious disease. In yet other embodiments, the pre-existing condition is further treated with the subject methods and compositions even after the end of illness caused by the infectious disease.

In some embodiments, the patient to be treated may develop organ or organ system dysfunction/failure while being treated for the infectious disease. For example, the patient may develop dysfunction or failure of one or more components of the cardiovascular system, respiratory system, renal system, haematological system, neurological system, gastrointestinal organs, hepatic organs, heart, liver, lungs, intestines, colon, kidneys, spleen, or brain while being treated for the infectious disease. In certain embodiments, the infectious disease causes the organ or organ system dysfunction/failure. For instance, the infectious disease may cause dysfunction or failure of one or more components of the cardiovascular system, respiratory system, renal system, haematological system, neurological system, gastrointestinal organs, hepatic organs, heart, liver, lungs, intestines, colon, kidneys, spleen, or brain in patients to be treated with the subject methods and compositions.

In certain embodiments, the patient may be a human subject that is being treated for respiratory illness, such as where the patient is being administered oxygen therapy. In some instances, the patient has been intubated. In certain instances, the patient is a human subject that is being administered oxygen therapy through the use of a mechanical breathing device, such as a ventilator. In some embodiments, methods may further include identifying a patient as in need of oxygen therapy, such as where the patient has been identified as having tissue damage to the lungs or has lung dysfunction or failure. In some instances, methods further include intubating the patient. In some instances, methods include administering oxygen therapy to the patient with a mechanical breathing device, such as a ventilator. In some cases, the patient is not on a ventilator.

In some embodiments, methods include identifying a subject as having an infectious disease. The subject may be identified or diagnosed as having the infectious disease by any suitable protocol, such as by a qualified healthcare provider. In some instances, identifying the subject as having an infectious disease includes determining that the subject exhibits one or more symptoms of the infectious disease, such as where a subject is identified as having a coronavirus infection by exhibiting one or more of a cough, respiratory distress or abnormal body temperature (e.g., a fever resulting in a body temperature of great than or equal to 100.4° F. or 100.8° F.). In some instances, the subject is identified as having an infectious disease through laboratory analysis of one or more fluid samples from the subject (e.g., through nasal or mucosal swab, saliva, blood). In certain instances, the subject is diagnosed as having an infectious disease through analysis by polymerase chain reaction (PCR). In some instances, the subject is identified as having an infectious disease through radiography, such as where respiratory disease is diagnosed in the subject through radiological analysis of the respiratory system (e.g., chest x-ray, ultrasound, computerized tomography (CT) scan, etc.). In some instances, the subject is identified as having an infectious disease by detecting antibodies to the infectious agent that causes the infectious disease.

In some cases, the subject has a mean arterial pressure (MAP) greater than or equal to 60 mm Hg, such as greater than or equal to 70 mm Hg, greater than or equal to 80 mm Hg, greater than or equal to 90 mm Hg, or greater than or equal to 100 mm Hg.

### Organ and/or Organ System Dysfunction and/or Failure

The methods generally include identifying or diagnosing subjects who are in need of treatment, e.g., subjects that would benefit from such treatment, e.g., due to being susceptible to organ dysfunction or failure, or already exhibiting at least one sign or symptom of organ dysfunction or failure. For example, the subject may be a member of a particular patient population such as those with disease resulting from acute insult (acute organ injury resulting from bacterial infection, severe burns, trauma, etc.), or chronic conditions (long-term exposure to organ-damaging medication), and/or from other causes which are discussed in more detail below. The patient to be treated may have, may not have, may have a history of, or may not have a history of organ dysfunction and/or failure.

The patient group(s) addressed by the present disclosure can also be defined as follows. The Sequential Organ Failure Assessment (SOFA) system was created in a consensus meeting of the European Society of Intensive Care Medicine in 1994 and further revised in 1996. The SOFA is a six-organ dysfunction/failure score measuring multiple organ failure daily. Each organ is graded from 0 (normal) to 4 (the most abnormal), providing a daily score of 0 to 24 points. The objective of the SOFA is to create a simple, reliable, and continuous score for clinical staff. Sequential assessment of organ dysfunction during the first few days of intensive care unit (ICU) or hospital admission is a good indicator of prognosis. In some embodiments, the mean and highest SOFA scores are used as predictors of outcome.

In a specific aspect, the patient group pursuant to the present disclosure is one having as a lower threshold at least one SOFA score, being at 1 for one of the clinical criteria of respiration, or liver, or coagulation, or cardiovascular, or CNS, or renal on the day of admission to hospital or ICU. Thus, said patient group is in need of therapeutic intervention pursuant to the present disclosure, and thus in need for prevention or reduction of organ dysfunction or organ failure.

In another specific aspect, the patient group pursuant to the present disclosure is one having as lower threshold at least two SOFA scores, being at 1 each for two of the clinical criteria respiration, and/or liver, and/or coagulation, and/or cardiovascular, and/or CNS, and/or renal on the day of admission to hospital or ICU. Thus, said patient group is in need of therapeutic intervention pursuant to the present disclosure, and thus in need for prevention or reduction of organ dysfunction or organ failure.

In another specific aspect, the patient group pursuant to the present disclosure is one having as a lower threshold at least three SOFA scores, being at 1 each for three of the clinical criteria respiration, and/or liver, and/or coagulation, and/or cardiovascular, and/or CNS, and/or renal on day of admission to hospital or ICU. Thus, said patient group is in need of therapeutic intervention pursuant to the present disclosure, and thus in need for prevention or reduction of organ dysfunction or organ failure.

In another specific aspect, the patient group pursuant to the present disclosure is one having as a lower threshold at least four SOFA scores, being at 1 each for four of the clinical criteria respiration, and/or liver, and/or coagulation, and/or cardiovascular, and/or CNS, and/or renal on the day of admission to hospital or ICU. Thus, said patient group is in need of therapeutic intervention pursuant to the present disclosure, and thus in need for prevention or reduction of organ dysfunction or organ failure.

In another specific embodiment, the patient group in need of prevention or reduction of renal organ dysfunction or renal organ failure pursuant to the present disclosure is having a renal SOFA score of at least 1, or of 2, or of 3, or of 4.

In another specific embodiment, the patient group in need of prevention or reduction of liver organ dysfunction or liver organ failure pursuant to the present disclosure is having a liver SOFA score of at least 1, or of 2, or of 3, or of 4.

In another specific embodiment, the patient group in need of prevention or reduction of heart organ dysfunction or heart organ failure pursuant to the present disclosure is having a cardiovascular SOFA score of at least 1, or of 2, or of 3, or of 4.

In another specific embodiment the patient group in need of prevention or reduction of lung organ dysfunction or lung organ failure pursuant to the present disclosure is having a respiratory SOFA score of at least 1, or of 2, or of 3, or of 4.

Independent of the initial score, generally an increase in SOFA score during the first 48 hours in the ICU or in the hospital predicts a mortality rate of at least 50%.

Thus, in another specific embodiment the patient group in need of therapeutic intervention for organ dysfunction/failure in accordance with present disclosure is characterized by having at least one SOFA score increased within the initial 48 hours after admission to hospital or ICU.

In some aspects, the organ, organs or organ systems which is/are subject to failure comprise at least one member of the following: cardiovascular, respiratory, renal, haematological, neurological, gastrointestinal organs, hepatic organs, heart, liver, lungs, intestines, colon, kidneys, spleen, and brain.

In some embodiments, the OCS and optionally at least one antimicrobial is to be used in combination with fluids administered intravenously, wherein said combination is for use in therapy of a subject having a chronic or acute disease or acute condition of a patient for protecting the organs of said patient. The fluids to be administered intravenously are, of course, administered systemically.

In one embodiment, the subject having a chronic or acute disease or condition being in need for protecting its organs is characterized by the need of the subject to receive intravenous fluids.

Unlike the optional at least one antimicrobial, the at least one OCS of the present disclosure may be administered for the sake of prevention or reduction of organ dysfunction and organ failure, and thus the at least one OCS is not necessarily intended for any methods of primary treatment or first line treatment of the chronic or acute disease or acute condition itself, which therefore can be termed as underlying disease(s).

### Kidney Dysfunction and/or Failure

The patient to be treated may have, may not have, may have a history of, or may not have a history of kidney disease, which may be acute or chronic, or even acute-on-chronic renal failure as discussed below.

Acute kidney injury (AKI, previously called acute renal failure (ARF)) refers to an abrupt loss of kidney function that develops, e.g., within about 7 days. AKI generally occurs because of damage to the kidney tissue caused by decreased renal blood flow (renal ischemia) from any cause, e.g., virus infection, low blood pressure, exposure to substances harmful to the kidney, an inflammatory process in the kidney, or an obstruction of the urinary tract which impedes the flow of urine. Causes of acute kidney injury include accidents, injuries, or complications from surgeries in which the kidneys are deprived of normal blood flow for extended periods of time. Heart-bypass surgery is an example of one such procedure. Drug overdoses, either accidental or from chemical overloads of drugs such as antibiotics or chemotherapy, may also cause the onset of acute kidney injury. AKI is in certain embodiments, diagnosed on the basis of characteristic laboratory findings, such as elevated blood urea nitrogen (BUN) and creatinine, or inability of the kidneys to produce sufficient amounts of urine (e.g., less than 400 mL per day in adults, less than 0.5 mL/kg/h in children or less than 1 mL/kg/h in infants). Thus, the present methods may include measuring or detecting one or more of these parameters in a subject and administering at least one OCS and optionally at least one antimicrobial to the subject, as described herein.

Chronic kidney disease (CKD) usually develops slowly and, initially, patients may show few symptoms. CKD can be the long term consequence of irreversible acute disease or part of a disease progression. CKD has numerous causes, including diabetes mellitus, long-term, uncontrolled hypertension, polycystic kidney disease, infectious diseases such as hantavirus, and certain genetic predisposition, e.g., APOL1 gene variants. The present methods include administering at least one OCS and optionally at least one antimicrobial to a subject having CKD.

In some cases, the clinical criteria denoting the patient group(s) for kidney dysfunction/failure are as follows:
Patients at risk for kidney dysfunction/failure: GFR decrease >25%, serum creatinine increased 1.5 times or urine production of <0.5 ml/kg/hr for 6 hours
Patients with present kidney injury: GFR decrease >50%, doubling of creatinine or urine production <0.5 ml/kg/hr for 12 hours
Patients with kidney failure: GFR decrease >75%, tripling of creatinine or creatinine >355 μmol/l (with a rise of >44 μmol/l) (>4 mg/dl) or urine output below 0.3 ml/kg/hr for 24 hours
Patients with loss of kidney function: persistent acute kidney injury (AKI) or complete loss of kidney function for more than 4 weeks
End-stage renal disease: complete loss of kidney function for more than 3 months.

In some cases, the patient has chronic kidney disease (CKD EPI eGFR between 15-45 mL/min/1.73 m²) or eGFR 45-59 mL/min/1.73 m² with acute liver injury (ALT>2×upper limit of population reference range).

In some cases, the patient has acute kidney injury stage 1, 2, 3, or 4 by Kidney Disease Improving Global Outcomes (KDIGO) criteria (i.e., at least doubling of serum creatinine from a known baseline value obtained within 12 months). In a preferred embodiment, the patient has (e.g., at least) stage 2 AKI by Kidney Disease Improving Global Outcomes (KDIGO) criteria.

The overuse of drugs such as aspirin, ibuprofen, and acetaminophen (paracetamol) can also cause chronic kidney disease. Contrast and enhancing dyes used for various types of imaging, especially iodine containing dyes, are also known to cause kidney damage, especially in susceptible populations such as the elderly, diabetics, those who already have some form of kidney impairment, etc. Contrast-induced nephropathy is defined as either a greater than 25% increase of serum creatinine or an absolute increase in serum creatinine of 0.5 mg/dL in the wake of administration of a dye, e.g., for X-rays or computed tomography (CT) scans. Iodine containing dyes include but are not limited to iohexol, iodixanol and ioversol, as well as other ionic iodine dyes such as Diatrizoate (Hypaque 50), Metrizoate (Isopaque 370), and Ioxaglate (Hexabrix); and non-ionic contrast media such as Iopamidol (Isovue 370), Iohexol (Omnipaque 350), Ioxilan (Oxilan 350), Iopromide (Ultravist 370), and Iodixanol (Visipaque 320).

### Liver Dysfunction and/or Failure

The patient to be treated may have, may not have, may have a history of, or may not have a history of liver dysfunction and/or failure. Acute liver failure involves the rapid development of hepatocellular dysfunction, specifically coagulopathy and mental status changes (encephalopathy) in a patient without known prior liver disease. This malady embraces a number of conditions whose common thread is severe injury of hepatocytes and/or massive necrosis, e.g., loss of function of 80-90% of liver cells. Loss of hepatocyte function sets in motion a multiorgan response characterized by the rapid appearance of severe complications soon after the first signs of liver disease (such as jaundice). Complications include hepatic encephalopathy and impaired protein synthesis, e.g., as measured by the levels of serum albumin and the prothrombin time in the blood. Up to now, treatment options for acute liver failure have been limited and death often occurs suddenly, even after the liver has begun to recover from the original damage.

The diagnosis of acute liver failure (i.e., the identification of subject experiencing acute liver failure and who could benefit from the practice of the present methods) is generally based on physical exam, laboratory findings, patient history, and past medical history to establish, for example, mental status changes, coagulopathy, rapidity of onset, and absence of known prior liver disease. The exact definition of “rapid” depends on the particular convention that is used. Different sub-divisions exist which are based on the time from onset of first hepatic symptoms to onset of encephalopathy. One scheme defines “acute hepatic failure” as the development of encephalopathy within 26 weeks of the onset of any hepatic symptoms. This is sub-divided into “fulminant hepatic failure”, which requires onset of encephalopathy within 8 weeks, and “subfulminant”, which describes onset of encephalopathy after 8 weeks but before 26 weeks. Another scheme defines “hyperacute” liver failure as onset within 7 days, “acute” liver failure as onset between 7 and 28 days, and “subacute” liver failure as onset between 28 days and 24 weeks. Subjects identified as experiencing acute liver failure by any of these criteria may be treated by the methods described herein.

In some cases, the patient group for liver dysfunction/failure is characterized by a lower threshold of bilirubin of >1.2 mg/dL, >1.9 mg/dL, >3.0 mg/dL, or >5.9 mg/dL.

In some cases, the patient to be treated has ALT>2×, >3×, >4×, >5×, >6×, >7×, >8×, >9×, or >10× upper limit of population reference range or upper limit of normal (ULN) of population reference range. In a preferred embodiment, the patient to be treated has acute liver injury or acute on chronic liver disease as defined by ALT>5×ULN or >3× baseline. In some cases, baseline ALT levels will not be known such that one skilled in the art would resort to comparison with ULN.

Acute liver failure has many potential causes and subjects identified as having a history of or experiencing acute liver failure for any reason can be treated by the methods described herein. Possible causes include:

Acetaminophen (ATMP). Taking too much acetaminophen (paracetamol, Tylenol®, others) is the most common cause of acute liver failure in the United States. Acute liver failure can occur if a single very large dose of ATMP is taken all at once, or it can occur if higher-than-recommended doses are taken every day for several days. People with chronic liver disease are especially vulnerable, as are the elderly, the very young, etc. In such subjects, an ATMP “overdose” may be a dose that would be a safe or normal dose for a person that does not have chronic liver disease or is not elderly or very young. This aspect of the disclosure is discussed in detail below.
Prescription medications. Some prescription medications, including antibiotics, nonsteroidal anti-inflammatory drugs and anticonvulsants, can cause acute liver failure.
Herbal supplements. Herbal drugs and supplements, including kava, ephedra, skullcap and pennyroyal, have been linked to acute liver failure.
Hepatitis and other viruses. Hepatitis A, hepatitis B and hepatitis E can cause acute liver failure. Other viruses that can cause acute liver failure include Epstein-Barr virus, cytomegalovirus, SARS-CoV-2, and herpes simplex virus.
Toxins. Toxins that can cause acute liver failure include the poisonous wild mushroom
Amanita phalloides, which is sometimes mistaken for edible species.
Autoimmune disease. Liver failure can be caused by autoimmune hepatitis, a disease in which the immune system attacks liver cells, causing inflammation and injury.
Diseases of the veins in the liver. Vascular diseases, such as Budd-Chiari syndrome, can cause blockages to form in the veins of the liver and lead to acute liver failure.
Metabolic disease. Rare metabolic diseases, such as Wilson's disease and acute fatty liver of pregnancy, can cause acute liver failure.
Cancer. Cancer that begins in the liver or cancer that spreads to the liver from other locations in the body can cause acute liver failure.
Other. Other causes include idiosyncratic reactions to medication (e.g., tetracycline, troglitazone), excessive alcohol intake (e.g., alcoholic hepatitis, such as severe alcoholic hepatitis), Reye syndrome (acute liver failure in a child with a viral infection, e.g., chickenpox in which aspirin may play a role); and others. Many cases of acute liver failure have no apparent cause.

In addition, various symptoms of liver toxicity may be prevented and/or treated by the methods and compositions of the present disclosure prior to the development of full-blown ALF. Symptoms may include but are not limited to: cerebral edema and encephalopathy (which may lead to hepatic encephalopathy, coma, brain herniation, etc.); coagulopathy (e.g., prolongation in prothrombin time, platelet dysfunction, thrombocytopenia, intracerebral bleeding, etc.); renal failure (e.g., due to original insult such as ATMP overdose resulting in acute tubular necrosis, or from hyperdynamic circulation leading to hepatorenal syndrome or functional renal failure); inflammation and infection (e.g., systemic inflammatory syndrome, which can lead to sepsis and multi- organ failure irrespective of the presence or absence of infection; various metabolic derangements such as hyponatremia, hypoglycemia, hypokalemia, hypophosphatemia, metabolic alkalosis, and lactic acidosis (occurring predominantly in acetaminophen overdose)); hemodynamic and cardio-respiratory compromise (e.g., hypotension, decrease in tissue oxygen uptake, tissue hypoxia and lactic acidosis); pulmonary complications (e.g., acute respiratory distress syndrome (ARDS), with or without sepsis, pulmonary haemorrhage, pleural effusions, atelectasis, and intrapulmonary shunts, etc.); late pregnancy complications, for which early clinical manifestations of ALF include hypodynamia, decrease in appetite, dark amber urine, deep jaundice, nausea, vomiting, and abdominal distention, etc. Subjects exhibiting one or more of these symptoms or conditions may benefit from the administration of at least one OCS and optionally at least one antimicrobial.

### Lung Dysfunction and/or Failure

The patients to be treated may have, may not have, may have a history of, or may not have a history of lung dysfunction and/or failure.

As used herein, the term “lung dysfunction” means all conditions having etiology based on or accompanied by insufficiency of gas exchange function in lungs, which are symptoms or diseases having connection with either disorder of permeation of oxygen contained in expired gas into resorptive epithelium, disorder of permeation from resorptive epithelium into blood via pulmonary capillary cells or disorder of uptake of oxygen into red cells (i.e., combination with hemoglobin). Examples of said symptoms or diseases include at least one of dyspnea or hypopnea resulted from physiologically active substance (such as narcotic, toxicant, etc.), foreign-body inhalation, COVID-19, anthracemia, bronchoconstriction attack in hypoxic condition (caused by smoke, dust, chemical irritant etc.), broncho-pulmonary injury, pulmonary contusion or shock, pulmonary edema, atelectasis, pulmonary thrombosis, pulmonary infarction, pulmonary fibrosis, pulmonary emphysema, bronchitis, bronchial asthma, adult respiratory distress syndrome (ADRS), infantile respiratory distress syndrome (IRDS), pulmonary stenosis, pulmonary congestion, pulmonary hypertension, chronic obstructive lung disease, congenital heart disease, bilateral carotid body enucleation, sudden infant death syndrome, uremia and central inhibition caused by narcotic or anesthetic.

In some cases, the patient has a lung condition comprising at least one of asthma, chronic obstructive pulmonary disease (COPD), cystic fibrosis (CF), allergic disorders, pulmonary inflammatory diseases, pulmonary fibrosis, and interstitial lung diseases.

By the term “respiratory dysfunction” is intended a clinically evident change in any of a number of physiologic parameters associated with normal lung function and respiration in mammals. Such parameters include, but are not limited to: tidal volume, vital capacity, forced expiratory volume, peak inspiratory pressure, spontaneous resting minute ventilation, VO₂, VCO₂, respiratory quotient, inspiratory muscle strength, lung elasticity, and diaphragm excursion.

In some cases, the patient has at least one of a vital capacity (<10-15 ml/kg); forced expiratory volume (<10 ml/kg/sec); peak inspiratory pressure (less than −20 to −30 cm H₂O); and spontaneous resting minute ventilation (<10 L/min).

In some embodiments, the patient has acute respiratory distress syndrome (ARDS), which is a rapidly progressive disorder that initially manifests as dyspnea, tachypnea, and hypoxemia, then quickly evolves into respiratory failure. In some cases, the subject has at least one of: acute onset; ratio of partial pressure of arterial oxygen to fraction of inspired oxygen (PaO₂/FiO₂) of 200 or less, regardless of positive end-expiratory pressure; bilateral infiltrates seen on frontal chest radiograph; and pulmonary artery wedge pressure of 18 mm Hg or less when measured, or no clinical evidence of left atrial hypertension.

### Sepsis

The patients to be treated may have, may not have, may have a history of, or may not have a history of sepsis.

Sepsis is a potentially life-threatening whole-body inflammation caused by a serious infection which triggers an immune response. The infection is typically caused by bacteria, but can also be due to fungi, viruses (e.g., SARS-CoV-2), or parasites in the blood, urinary tract, lungs, skin, or other tissues. Unfortunately, symptoms can continue even after the infection is gone. Severe sepsis is sepsis causing poor organ function or insufficient blood flow as evidenced, e.g., by low blood pressure, high blood lactate, and/or low urine output. In fact, sepsis is considered to fall within a continuum from infection to multiple organ dysfunction syndrome (MODS). Septic shock is low blood pressure due to sepsis that does not improve after reasonable amounts of intravenous fluids are given.

Up to now, sepsis was typically treated with intravenous fluids and antibiotics, often in an intensive care unit. Various medications and other interventions may be used, e.g., mechanical ventilation, dialysis, and oxygen saturation may also be used. Outcomes depend on the severity of disease with the risk of death from sepsis being as high as 30%, severe sepsis as high as 50%, and septic shock as high as 80%.

Sepsis is associated with mitochondrial dysfunction, which leads to impaired oxygen consumption and may lead to sepsis-induced multiple organ failure. This holds especially true for raised tissue oxygen tensions in septic patients, suggesting reduced ability of the organs to use oxygen. Because ATP production by mitochondrial oxidative phosphorylation accounts for more than 90% of total oxygen consumption, mitochondrial dysfunction may directly result in organ failure, possibly due to nitric oxide, which is known to inhibit mitochondrial respiration in vitro and is produced in excess in sepsis. Therefore, in some embodiments of the present disclosure, the at least one OCS and optionally at least one antimicrobial are used in methods of prevention for organ dysfunction and failure in Systemic Inflammatory Response-Syndrome (SIRS), sepsis, severe sepsis, and septic shock patients.

The methods may include identifying a suitable patient in need of such treatment, e.g., by detecting or measuring at least one symptom of sepsis, e.g., abnormal temperature (body temperature above 100.8° F. (38.3° C., “fever”) or below 96.8° F. (36° C.), increased heart rate, increased breathing rate, probable or confirmed infection, and possibly confusion. Patients with severe sepsis exhibit at least one of the following signs and symptoms, which indicate an organ may be failing: significantly decreased urine output, abrupt change in mental status, decrease in platelet count, difficulty breathing, abnormal heart pumping function, and abdominal pain. A diagnosis of septic shock is generally based on observing the signs and symptoms of severe sepsis plus measuring extremely low blood pressure that does not adequately respond to simple fluid replacement.

In some cases, a subject may be a candidate for prophylactic or therapeutic treatment with at least one OCS and optionally at least one antimicrobial of sepsis is based on cough/sputum/chest pain; abdominal pain/distension/diarrhea; line infection; endocarditis; dysuria; headache with neck stiffness; cellulitis/wound/joint infection; and/or positive microbiology for any infection.

In other cases, a subject may be a candidate for prophylactic or therapeutic treatment with at least one OCS and optionally at least one antimicrobial of severe sepsis based on a diagnosis of sepsis and at least one clinical suspicion of any organ dysfunction selected from: blood pressure systolic <90/mean; <65 mm HG; lactate >2 mmol/L; bilirubin >34 μmol/L; urine output <0.5 mL/kg/h for 2 h; creatinine >177 μmol/L; platelets <100×10⁹/L; and SpO₂>90% unless O₂ given.

In some cases, the subject has infection plus at least one of the following findings: temperature >100.9° F. (38.3° C.) or <96.8° F. (36° C.); pulse >90 beats per minute; tachypnea; altered mental status; white blood cell count >12,000 per mm³ (12×10⁹ per L), <4,000 mm³ (4×10⁹ per L), or >10 percent immature forms; elevated C-reactive protein level; arterial hypotension; acute oliguria; and/or hyperlactatemia.

In some cases, a subject may be a candidate for prophylactic or therapeutic treatment with at least one OCS and optionally at least one antimicrobial of septic shock if there is refractory hypotension that does not respond to treatment and intravenous systemic fluid administration alone is insufficient to maintain a patient's blood pressure from becoming hypotensive.

Patients with a diagnosis of (exhibiting signs of) early sepsis, severe sepsis or septic shock are candidates for treatment with the at least one OCS and optionally at least one antimicrobial described herein, e.g., by administration of a therapeutically effective amount of at least one OCS as described herein (e.g., 25HC3S). The amount administered may be sufficient to prevent symptoms of sepsis from developing or continuing, or to at least lessen the impact of symptoms of sepsis.

### Pancreas Dysfunction and/or Failure

In some cases, the patient has or has a history of pancreas dysfunction and/or failure.

The pancreas is a glandular organ that functions in the digestive system and endocrine system of vertebrates. It produces several important hormones, including insulin, glucagon, somatostatin, and pancreatic polypeptide, and also secretes pancreatic juice containing digestive enzymes that assist digestion and absorption of nutrients in the small intestine. Inflammation of the pancreas (pancreatitis) has several causes, such as viral (e.g., SARS-CoV-2), and typically requires immediate treatment. It may be acute, beginning suddenly and lasting a few days, or chronic, occurring over many years. Eighty percent of cases of pancreatitis are caused by alcohol or gallstones, with gallstones being the single most common etiology of acute pancreatitis and alcohol being the single most common etiology of chronic pancreatitis. Severe pancreatitis is associated with organ failure, necrosis, infected necrosis, pseudocyst and abscess, having mortality rates around 2-9%, and higher where necrosis has occurred. The subject may have at least one of (and severe pancreatitis may be diagnosed if at least three of the following are true): patient age is greater than 55 years; blood PO₂ oxygen is less than 60 mm Hg or 7.9 kP; white blood cells>15,000 WBCs per microliter (mcL); calcium<2 mmol/L; urea>16 mmol/L; lactate dehydrogenase (LDH)>600 iu/L; aspartate transaminase (AST)>200 iu/L; albumi<32 g/L; and glucose>10 mmol/L.

An aspect of the present disclosure is the treatment of pancreatic dysfunction and/or failure by administering at least one OCS and optionally at least one antimicrobial to a patient in need thereof.

### Heart Dysfunction and/or Failure

In some cases, the patient has or has a history of heart dysfunction and/or failure. Heart failure (HF), often used to mean chronic heart failure (CHF), occurs when the heart is unable to pump sufficiently to maintain blood flow to meet the needs of the body. The terms congestive heart failure (CHF) or congestive cardiac failure (CCF) are often used interchangeably with chronic heart failure. Symptoms commonly include shortness of breath (especially with exercise, when lying down, and at night while sleeping), excessive tiredness, and leg swelling. Common causes of heart failure include coronary artery disease including a previous myocardial infarction (heart attack), high blood pressure, atrial fibrillation, valvular heart disease, viruses (e.g., SARS-CoV-2), and cardiomyopathy. Heart failure is distinct from myocardial infarction, in which part of the heart muscle dies, and cardiac arrest, in which blood flow stops altogether.

Heart failure is typically diagnosed based on the history of the symptoms and a physical examination with confirmation by echocardiography, blood tests, and/or chest radiography. Echocardiography uses ultrasound to determine the stroke volume (SV, the amount of blood in the heart that exits the ventricles with each beat), the end-diastolic volume (EDV, the total amount of blood at the end of diastole), and the SV in proportion to the EDV, a value known as the ejection fraction (EF). Abnormalities in one or more of these may indicate or confirm heart dysfunction and/or failure. An electrocardiogram (ECG/EKG) is used to identify arrhythmias, ischemic heart disease, right and left ventricular hypertrophy, and presence of conduction delay or abnormalities (e.g., left bundle branch block). Abnormalities in one or more of these may also indicate or confirm heart dysfunction and/or failure. Blood tests routinely performed to diagnose or confirm heart dysfunction/failure include electrolytes (sodium, potassium), measures of renal function, liver function tests, thyroid function tests, a complete blood count, and often C-reactive protein if infection is suspected. Abnormalities in one or more of these may also indicate or confirm the presence of heart dysfunction and/or failure. An elevated B-type natriuretic peptide (BNP) is a specific test indicative of heart failure. If myocardial infarction is suspected, various cardiac markers may be tested, including but not limited to troponin creatine kinase (CK)-MB (an isoform of creatine kinase); lactate dehydrogenase; aspartate transaminase (AST) (also referred to as aspartate aminotransferase); myoglobin; ischemia-modified albumin (IMA); pro-brain natriuretic peptide; glycogen phosphorylase isoenzyme BB, etc. Abnormal levels of one or more of these (usually abnormally high levels) are considered as identifying a subject in need of treatment for cardiac dysfunction or failure. In some cases, the subject has a troponin level greater than 0.04 ng/mL, such as greater than 0.1 ng/mL, greater than 1 ng/mL, greater than 10 ng/mL, greater than 20 ng/mL, greater than 30 ng/mL, greater than 40 ng/mL, or greater than 50 ng/mL, and may range from about 0.04 ng/mL and about 150 ng/mL, such as about 0.08 ng/mL to about 100 ng/mL or about 0.1 ng/mL to about 80 ng/mL,

A subject who is confirmed to have or suspected of having cardiac dysfunction or failure is treated by administration of a therapeutically effective amount of at least one OCS as described herein (e.g., 25HC3S) and optionally at least one antimicrobial, the amount being sufficient to prevent symptoms of heart dysfunction or failure, or to ameliorate symptoms of heart dysfunction or failure, e.g., to at least partially restore heart function to normal or near normal, and/or to prevent further deterioration of heart function and health of the patient.

### Brain Dysfunction and/or Failure

In some cases, the patient has or has a history of brain dysfunction and/or failure.

Brain dysfunction and/or failure (i.e., organic brain syndrome “OBS”) is a general term that describes decreased mental function due to a medical disease other than a psychiatric illness. Causes include but are not limited to brain injury caused by trauma; bleeding into the brain (intracerebral hemorrhage); bleeding into the space around the brain (subarachnoid hemorrhage); blood clot inside the skull causing pressure on brain (subdural hematoma); concussion; various breathing conditions such as low oxygen in the body (hypoxia) and high carbon dioxide levels in the body (hypercapnia); various cardiovascular disorders, e.g., dementia due to many strokes or multi-infarct dementia, heart infections (endocarditis, myocarditis), stroke (e.g., spontaneous stroke) and transient ischemic attack (TIA) or so-called “ministrokes”; or due to various degenerative disorders such as Alzheimer disease, Creutzfeldt-Jacob disease, diffuse Lewy Body disease, Huntington disease, multiple sclerosis, normal pressure hydrocephalus, Parkinson disease and Pick disease; dementia due to metabolic causes such as kidney, liver, or thyroid disease and/or vitamin deficiency (B1, B12, or folate); as well as drug and alcohol-related conditions, e.g., alcohol withdrawal state, intoxication from drug or alcohol use, Wernicke-Korsakoff syndrome (a long-term effect of excessive alcohol consumption or malnutrition), and withdrawal from drugs (especially sedative-hypnotics and corticosteroids); and sudden onset (acute) or long-term (chronic) infections, e.g., septicemia, encephalitis, meningitis, prion infections, and late-stage syphilis; as well as complications of cancer or cancer treatment. Symptoms of OBS include agitation, confusion; long-term loss of brain function (dementia), and severe, short-term loss of brain function (delirium), as well as impacts on the autonomic nervous system which controls, e.g., breathing. Diagnosis or confirmation of the presence of OBS is determined by detecting or measuring various methodology such as blood tests, electroencephalogram (EEG), head CT scan, head MRI and/or lumbar puncture (for which normal values typically range as follows: pressure: 70-180 mm Hg; cerebral spinal fluid (CSF) appearance: clear, colorless; CSF total protein: 15-60 mg/100 mL; gamma globulin: 3-12% of the total protein; CSF glucose: 50-80 mg/100 mL (or greater than ⅔ of blood sugar level); CSF cell count: 0-5 white blood cells (all mononuclear), and no red blood cells; and CSF chloride: 110-125 mEq/L).

If one or more of these tests or analyses or indicia are abnormal, the subject is generally considered as susceptible to or already suffering from OBS. A subject who is confirmed to have or suspected of having OBS (either early stage or advanced) is treated by administration of a therapeutically effective amount of at least one OCS as described herein (e.g., 25HC3S) and optionally at least one antimicrobial, the amount being sufficient to prevent symptoms of OBS, or to ameliorate symptoms of OBS, e.g., to at least partially restore brain function to normal or near normal, and/or to prevent further deterioration of brain function and health of the patient.

### Trauma

In some cases, the patient has or has a history of organ dysfunction/failure due to trauma.

Examples of trauma injuries include but are not limited to: wounds resulting from vehicular accidents; gunshot wounds (both accidental during hunting associated activities, and intentionally inflicted such as those associated with criminal activity or war); blunt trauma or blunt injury, e.g., non-penetrating blunt force trauma such as physical trauma to a body part, e.g., by impact, injury or physical attack; etc. Examples of blunt trauma include but are not limited to: concussion, e.g., concussion suffered by athletes or by persons involved in accidents, falls, etc., and blunt trauma suffered as the result of an encounter with a projectile such as a falling object, and others.

Individuals who are susceptible to such blunt trauma (e.g., athletes, the elderly) may benefit from prophylactic administration of one or more OCS, and if blunt trauma such as a concussion is diagnosed in a subject, the subject will benefit by administration as soon as possible after the injury is suspected or confirmed.

### Ischemia

In some case, the patient has or has a history of ischemia.

Ischemia refers to an insufficient supply of blood to a tissue or organ, causing a shortage of oxygen and glucose needed for cellular metabolism and to keep tissue alive. Hypoxia (also known as hypoxiation or anoxemia) is caused by ischemia and refers to the condition in which the body or a region of the body is deprived of adequate oxygen supply. Ischemia results in tissue damage in a process known as the ischemic cascade. Damage is largely the result of the build-up of metabolic waste products, the inability to maintain cell membranes, mitochondrial damage, and eventual leakage of autolyzing proteolytic enzymes into the cell and surrounding tissues. Ensuing inflammation also damages cells and tissues. Without immediate intervention, ischemia may progress quickly to tissue necrosis, and ultimately to, for example, organ dysfunction or failure.

In addition, restoration of blood supply to ischemic tissues can cause additional damage known as reperfusion injury. Reperfusion injury can be more damaging than the initial ischemia. Reintroduction of blood flow brings oxygen back to the tissues, causing a greater production of free radicals and reactive oxygen species that damage cells. It also brings more calcium ions to the tissues, which may cause calcium overloading and can result in potentially fatal cardiac arrhythmias, and which may accelerate cellular self-destruction. The restored blood flow may also exaggerate the inflammation response of damaged tissues, causing white blood cells to destroy damaged but still viable cells.

The present disclosure provides methods of preventing and/or treating the untoward effects or outcomes of ischemia, including ischemia/reperfusion injury, in a subject in need thereof. The methods may comprise administering a therapeutically effective amount of one or more OCS and optionally at least one antimicrobial sufficient to prevent or treat symptoms of ischemia and/or ischemia/reperfusion. The methods may also include identifying or diagnosing a subject who will experience, or is experiencing or who has experienced ischemia and/or ischemia/reperfusion. The ischemia and/or ischemia/reperfusion may be due to a disease process (e.g., arthrosclerosis, a blood clot, etc.), due to a virus (e.g., SARS-CoV-2), due to an accident (e.g., severing of an artery or other blood conduit), or may be intentional (planned), e.g., as occurs during some heart or other surgeries in order to temporarily stop blood flow to a defined or circumscribed region of the body.

Types of ischemia that are relevant to the methods described herein include but are not limited to:

Cardiac ischemia, e.g., myocardial ischemia, occurring when the heart muscle, or myocardium, receives insufficient blood flow. This most frequently results from atherosclerosis, which is the long-term accumulation of cholesterol-rich plaques in coronary arteries.
Bowel ischemia: Both large and small bowel can be affected by ischemic injury. Ischemic injury of the large intestine may result in an inflammatory process known as ischemic colitis and also as a result of surgery and adhesion development. Ischemia of the small bowel is called mesenteric ischemia.
Brain ischemia is insufficient blood flow to the brain, and can be acute (i.e., rapid) or chronic (i.e., long-lasting). Acute ischemic stroke is a neurologic emergency that may be reversible if treated rapidly. Chronic ischemia of the brain may result in a form of dementia called vascular dementia. A brief episode of ischemia affecting the brain is called a transient ischemic attack (TIA), often referred to as a “mini-stroke”.
Limb ischemia: Lack of blood flow to a limb results in acute limb ischemia.
Cutaneous ischemia refers to reduced blood flow to the skin layers, which may result in mottling or uneven, patchy discoloration of the skin, and may lead to the development of cyanosis, or other conditions such as pressures sores (e.g., decubitus ulcers, bedsores, etc.).
Reversible ischemia refers to a condition which results in a lack of blood flow to a particular organ which can be reversed through use of medications or surgery. It most often refers to hindered blood flow to the heart muscle, but it can refer to an obstruction blocking any organ in the body, including the brain. Whether or not a case of ischemia can be reversed will depend on the underlying cause. Plaque buildup in the arteries, weakened arteries, low blood pressure, blood clots, and unusual heart rhythms can all be causes of reversible ischemia.
Apical ischemia refers to lack of blood flow to the apex or bottom tip of the heart. Mesenteric ischemia refers to inflammation and injury of the small intestine occurs due to inadequate blood supply. Causes of the reduced blood flow can include changes in the systemic circulation (e.g., low blood pressure) or local factors such as constriction of blood vessels or a blood clot.
Ischemia of various organs, including but not limited to liver (hepatic ischemia), kidney, intestines, etc.

Ischemia, ischemia/reperfusion may also be causally related to inflammation and organ dysfunction/failure. For example, cerebral (brain) ischemia is typically accompanied by a marked inflammatory reaction that is initiated by ischemia-induced expression of cytokines, adhesion molecules, and other inflammatory mediators, including prostanoids and nitric oxide. It is known that interventions aimed at attenuating such inflammation reduce the progression of brain damage that occurs, e.g., during the late stages of cerebral ischemia. In addition, the most frequent cause of intrarenal (kidney) failure (ARF) is transient or prolonged renal hypoperfusion (ischemia).

Other types of ischemia, the effects of which can be treated or prevented as described herein, include but are not limited to: ischemic stroke, small vessel ischemia, ischemia/reperfusion injuries, etc.

Diagnosis of ischemia is generally carried out by identifying one or more symptoms of malfunction in the particular organ or organ system or tissue or cell that is affected. Thus, symptoms include those listed herein for dysfunction/failure of individual organs, plus documentation of ischemia per se, such as by noting the history of the patient (e.g., known occlusion, blockage or severance of an artery that otherwise supplies blood to the organ or tissue, imaging which shows or is consistent with such observations,

If one or more suitable tests or analyses or indicia are abnormal, the subject is generally considered as susceptible to or already suffering from ischemia. A subject who is confirmed to have or suspected of having ischemia (or is known to be undergoing future planned ischemia, e.g., during a surgical procedure) may be treated by administration of a therapeutically effective amount of at least one OCS as described herein (e.g., 25HC3S) and optionally at least one antimicrobial, the amount being sufficient to prevent symptoms of ischemia and/or ischemia-reperfusion injury, or to ameliorate symptoms of ischemia and/or ischemia-reperfusion injury, e.g., to at least partially restore organ or tissue function to normal or near normal when blood flow is reestablished, and/or to prevent further deterioration of organ or tissue function and health of the patient.

### DESCRIPTION OF ADMINISTRATION

Implementation of the methods generally involves identifying patients suffering from an infectious disease and administering at least one oxygenated cholesterol sulfate (OCS) and optionally at least one antimicrobial in an acceptable form by an appropriate route. The exact total amount to be administered may vary depending on the age, gender, weight and overall health status of the individual patient, as well as the precise etiology of the disease.

Examples of the at least one OCS that are used in the methods and compositions described herein include but are not limited to 5-cholesten-3, 25-diol, 3-sulfate (25HC3S); 5-cholesten, 3, 25-diol, disulfate (25HCDS); (5-cholestene, 3, 27-diol, 3-sulfate); (5-cholestene, 3, 27-diol, 3, 27-disulfate); (5-cholestene, 3,7-diol, 3-sulfate); (5-cholestene, 3,7-diol, 3,7-disulfate); (5-cholestene, 3, 24-diol, 3-sulfate); (5-cholestene, 3, 24-diol, 3, 24-disulfate); (5-cholestene, 3-ol, 24, 25-epoxy 3-sulfate); and salts thereof. Disclosure of 25HC3S is found in, e.g., U.S. Pat. No. 8,399,441, which is incorporated herein by reference in its entirety. Disclosure of 25HCDS is found, e.g., in U.S. Published Application No. 20150072962, which is incorporated herein by reference in its entirety. In certain aspects, the at least one OCS is selected from 5-cholesten-3, 25-diol, 3-sulfate (25HC3S) and 5-cholesten, 3, 25-diol, disulfate (25HCDS) (either alone or in combination), or salts thereof. In further aspects, the at least one OCS is 5-cholesten-3, 25-diol, 3-sulfate (25HC3S) or salt thereof.

In some embodiments, the total amount of the at least one OCS administered (e.g., in a period of one hour, one day, one week, or one month) typically ranges from about 0.01 mg/kg to about 50 mg/kg, more usually from about 0.05 mg/kg to about 20 mg/kg, such as from about 0.1 mg/kg to about 10 mg/kg, from about 0.2 mg/kg to about 6 mg/kg, from about 0.2 mg/kg to about 2 mg/kg, or from about 0.3 mg/kg to about 1.5 mg/kg, of at least one OCS per kg of body weight.

Total amounts of the at least one OCS generally range from about 1 mg to about 1000 mg of the at least one OCS. Typically, in some embodiments, the total amount of the at least one OCS administered to the patient in the methods (e.g., in a period of one day, one week, or one month) is from about 10 mg to about 500 mg, such as about 50 mg to about 400 mg. For instance, in some embodiments, the total amount is at least about 20 mg. In some embodiments, the total amount is not more than about 500 mg, such as not more than about 400 mg, not more than about 300 mg, not more than about 200 mg, or even not more than about 140 mg. Particular examples of total amounts administered include from about 10 mg to about 600 mg, about 10 mg to about 400 mg, from about 20 mg to about 300 mg, from about 20 mg to about 200 mg, and from about 40 mg to about 140 mg. The total amount will vary with the route of administration, the bioavailability, and the particular formulation that is administered. For instance, for intravenous administration, the total amounts described herein may be particularly useful.

In some embodiments, at least one, and preferably each, of the one or more separate doses comprises from about 10 mg to about 300 mg of the at least one OCS. For instance, in some embodiments at least one, and preferably each, of the one or more separate doses comprises from about 10 mg to about 200 mg of the at least one OCS, such as from about 20 mg to about 150 mg, about 20 mg to about 120 mg, or about 20 mg to about 100 mg of the at least one OCS. In some embodiments, particular useful numbers of separate doses for such dose amounts may be one dose (i.e., a single dose) or two separate doses.

In view of the above, the total amount of the at least one OCS administered in a period of one month may range from about 0.01 mg/kg/month to about 50 mg/kg/month, more usually from about 0.05 mg/kg/month to about 20 mg/kg/month, such as from about 0.1 mg/kg/month to about 10 mg/kg/month, e.g., from about 0.2 mg/kg/month to about 6 mg/kg/month or from about 0.3 mg/kg/month to about 1.5 mg/kg/month. The total amount of the at least one OCS or salt thereof administered in a period of one month may range from about 1 mg/month to about 1000 mg/month, from about 5 mg/month to about 600 mg/month, from about 10 mg/month to about 400 mg/month, from about 20 mg/month to about 300 mg/month, from about 20 mg/month to about 200 mg/month, or from about 20 mg/month to about 140 mg/month.

The optional antimicrobial may comprise at least one antiviral, at least one antifungal, at least one antiparasitic, and/or at least one antibacterial.

In some cases, the antimicrobial comprises at least one of an HIV drug, an Ebola drug, an anti-influenza drug, an anti-parainfluenza drug, an Respiratory Syncytial Virus (RSV) drug, an anti-hepatitis C vaccine (anti-HCV), an antibiotic, a biologic, an antibody, an immunomodulator, an anti-inflammatory, an IL-6 inhibitor, a drug that impedes trimerization of SARS-CoV-2 spike glycoprotein and inhibits host cell adhesion, a protease inhibitor, a nucleoside analog, a purine nucleoside analog, a nucleoside inhibitor of RNA polymerase, a small interfering RNA (siRNA), an anticoagulant, an antipancreatitis drug, and a TMPRSS2 protease inhibitor.

In some cases, the antiviral comprises at least one of Neumifil, Ribavirin, Fenretinide, Favipiravir (a.k.a. Favilavir) (e.g., Avigan™ (favipiravir, T-705)), Brincidofovir (nucleotide analogue cidofovir), ZMapp, TKM-100802, BCX4430, Interferons (e.g., Interferon-β-1α, Peginterferon lambda-1a, and Interferon β-1b, such as Ribavirin with Interferon β-1b), Bamlanivimab, anakinra (e.g., Kineret®), Lenzilumab, at least one of casirivimab and imdevimab (e.g., REGN-COV2), at least one of MP0420 and MP0423, Amiodarone, Atorvostatin, Irbesartan, Clomiphene, FX06, Zmab, Gimsilumab, I-Mab, OYA1, Chlorpromazine, Loperamide, BPI-002, APN01, Brilacidin, Leronlimab (PRO 140), Galidesivir (BCX4430), REGN3048, REGN3051, SNG001, Saquinavir, Indinavir, Carfilzomib, Novaferon, danoprevir (e.g., Ganovo® danoprevir), ASC09, oseltamivir (e.g., Tamiflu® oseltamivir), IFX-1, CYNK-001, acyclovir (e.g., Zovirax® acyclovir), famcyclovir (e.g., Famvir® famcyclovir), valcyclovir (e.g., Valtrex® valcyclovir), mercaptopurine, emodin, toremifene, tamoxifen (e.g., Novaldex® tamoxifen), Albendazole, AC-93253, Toremifene, at least one of lopinavir and ritonavir (e.g., Kaletra® lopinavir/ritonavir), rilpivirine, dolutegravir, raltegravir, elvitegravir, zalcitabine, molnupiravir (EIDD-2801, MK-4482), nafamostat, at least one of sofosbuvir and daclatasvir, at least one of sofosbuvir and velpatasvir, at least one of darunavir and cobicistat (e.g., PREZCOBIX® darunavir/cobicistat), umifenovir (e.g., Arbidol™ umifenovir), and GS-5734 (remdesivir).

In some cases, the at least one antifungal comprises at least one of Ciclopirox, Ciclopirox Olamine, clotrimazole, miconazole, ketoconazole, econazole, terconazole, tioconazole, sertaconazole, butoconazole, oxiconazole, sulconazole, metronidazole, posoconazole, terconazole, itraconazole, fluconazole, sirolimus, dactinomycin, Terbinafine, neftifine, butenafine, Nystatin, Amphotericin B, Haloprogin, Griseofulvin, and a Benzoxaborole.

In some cases, the at least one antiparasitic comprises at least one of atipamezole, abamectin, ivermectin, Avrmectin, Moxidectin, emamectin, Eprinomectin, selamectin, doramectin, Nemadectin, albendazole, cambendazole, fenbendazole, flubendazole, mebendazole, oxfendazole, oxibendazole, parbendazole, tetramisole, levamisole HC1, pyrantel embonate, oxantel, Morantel, indoxacarb, closantel, triclabendazole, clorsulon, rafoxanide, niclosamide, praziquantel, epsiprantel, paraherquamides, pyripole, pyrazine ethiprole, lufenuron, Spiromesifen, worm hydrazides, pleocidin, ethyl pleocidin, Provado, MTI-446, metaflumizone, thibendiamide, chlorantraniliprole, pyridalyl, Deltamethrin, pyrimidifen, fluorine worm pyrrole quinoline, CGA-179246, milbemycin, demiditraz, amitraz, ethiprole, S-methoprene, hydroprene, Nylar, permethrin, pyrethrin, e.g. synthetic pyrethroids (e.g., cypermethrin, lambdacyhalothrin, fenvalerate, resmethrin, tralomethrin, cyphenotrin), acetylcholinesterase inhibitors as carbamates (e.g., carbaryl, benziocarb, fenoxycarb, proxopur), acetylcholine mimics (e.g., nicotine, imidacloprid), neonicotinoid compounds (e.g., nitenpyram), and GABA antagonists (e.g., fipronil).

In some cases, the at least one antibacterial comprises at least one of actinomycins, aminoglycosides, beta-lactamase inhibitors, glycopeptides, ansamycins, bacitracins, carbacephems, carbapenems, cephalosporins, isoniazid, linezolid, macrolides, mupirocin, penicillins, oxolinic acid, polypeptides, quinolones, sulfonamides, tetracyclines, monobactams, chloramphenicol, lincomycin, clindamycin, ethambutol, mupirocin, metronidazole, pefloxacin, pyrazinamide, thiamphenicol, rifampicin, thiamphenicol, dapsone, clofazimine, quinupristin, metronidazole, linezolid, isoniazid, piracil, novobiocin, trimethoprim, fosfomycin, fusidic acid, scefazolin, gentamicin, and ampicillin.

The administration of the the at least one OCS and optionally at least one antimicrobial may be intermittent, or at a gradual or continuous, constant or controlled rate.

Administration may be through any route, such as parenteral, including injection intravenously, intramuscularly, and/or subcutaneously. The route of administration will depend on the nature of the condition that is treated, e.g., on the type or degree of organ injury or failure, such as liver injury and/or liver failure. For example, to achieve expedited treatment before significant organ injury or failure, such as liver dysfunction or failure, has occurred, dosing by intravenous injection may be preferred. Thus, when damage has already occurred, and especially when acute organ failure is diagnosed, the route of administration is generally parenteral or intravenous to speed delivery of the at least one OCS and optionally at least one antimicrobial.

The at least one OCS and optionally at least one antimicrobial may be administered in the pure form or in a pharmaceutically acceptable formulation including suitable elixirs and the like (generally referred to as “carriers”) or as pharmaceutically acceptable salts (e.g., alkali metal salts such as sodium, potassium, calcium or lithium salts, ammonium, etc.) or other complexes. It may, for instance, be preferable to utilize a salt of the at least one OCS and/or the optional at least one antimicrobial; the sodium salt of 25HC3S is one exemplary such salt. It should be understood that the pharmaceutically acceptable formulations may include liquid materials conventionally utilized to prepare injectable dosage forms. The at least one OCS and optionally at least one antimicrobial are typically administered as compositions that are liquids suitable for injection and/or intravenous administration. Solid forms suitable for solution in, or suspension in, liquids prior to administration may also be prepared.

The active ingredients may be mixed with excipients which are pharmaceutically acceptable and compatible with the active ingredients, e.g., pharmaceutically and physiologically acceptable carriers. Suitable excipients include, for example, water, saline (sodium chloride), cyclodextrin (e.g., hydroxypropyl-beta-cyclodextrin), dextrose, glycerol, ethanol and the like, or combinations thereof. In addition, the composition may contain minor amounts of auxiliary substances such as wetting or emulsifying agents, pH buffering agents (e.g., phosphate buffer), and the like. Water may be used as the carrier for the preparation of compositions (e.g., injectable compositions), which may also include conventional buffers and agents to render the composition isotonic. Other potential additives and other materials (preferably those which are generally regarded as safe [GRAS]) include: surfactants (TWEEN®, oleic acid, etc.); solvents, stabilizers, elixirs, and encapsulants (lactose, liposomes, etc). Preservatives such as methyl paraben or benzalkium chloride may also be used. The composition of the present disclosure may contain any such additional ingredients so as to provide the composition in a form suitable for the intended route of administration. In addition, the compounds may be formulated with aqueous or oil based vehicles.

Depending on the formulation, it is expected that the at least one OCS and optionally at least one antimicrobial will be present at about 1 wt % to about 99 wt % of the composition and the vehicular “carrier” will constitute about 1 wt % to about 99 wt % of the composition. The pharmaceutical compositions of the present disclosure may include any suitable pharmaceutically acceptable additives or adjuncts to the extent that they do not hinder or interfere with the therapeutic effect of the at least one OCS and optionally at least one antimicrobial.

The at least one antimicrobial of the present disclosure may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations typically range from about 3 to about 11, but is ordinarily about 7 to 10. In some embodiments, the pH of the formulations ranges from about 2 to about 5, but is ordinarily about 3 to 4.

While it is possible for the at least one OCS and optionally at least one antimicrobial to be administered alone it may be preferable to present them as pharmaceutical formulations. The formulations, both for veterinary and for human use, of the present disclosure comprise at least one active ingredient, as above defined, together with one or more acceptable carriers therefor and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present disclosure suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues, e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this disclosure may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the present disclosure include Tween® 60 polyethylene glycol sorbitan monostearate, Span® 80 sorbitan monooleate, cetostearyl alcohol, benzyl alcohol, myristyl alcohol, glyceryl mono-stearate and sodium lauryl sulfate. Further emulgents and emulsion stabilizers suitable for use in the formulation of the present disclosure include Tween® 80 sorbitan monooleate.

The choice of suitable oils or fats for the formulations may be based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present disclosure may comprise a combination of the at least one OCS and optional at least one antimicrobial together with one or more pharmaceutically acceptable carriers or excipients and optionally other therapeutic agents. Pharmaceutical formulations containing the active ingredient(s) may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the present disclosure may contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally- occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin and Captisol (=Sulfobutyl ether beta-cyclodextrin; SEB-beta-CD).

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the present disclosure suitable for preparation of an aqueous suspension by the addition of water may provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the present disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 1000 μg to about 2000 μg or about 3 μg to about 500 μg of the active ingredient per milliliter of solution to facilitate infusion at a suitable rate, e.g., ranging from 20 mL/hr to 100 mL/hr, such as a rate of about 50 mL/hr or about 30 mL/hr.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described below.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration may include aqueous and non- aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this disclosure may include other agents conventional in the art having regard to the type of formulation in question, for example those suitable for oral administration may include flavoring agents.

The present disclosure further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the present disclosure may be used to provide controlled release pharmaceutical formulations containing as active ingredient one or more compounds of the present disclosure (“controlled release formulations”) in which the release of the active ingredient are controlled and regulated to allow less frequent dosing or to improve the pharmacokinetic or toxicity profile of a given active ingredient.

Compositions of the present disclosure are also used in combination with other active ingredients. Non-limiting examples of these other active therapeutic agents are a steroid, a rheumatoid arthritis drug, a Janus kinase inhibitor, and an angiotensin receptor blocker. Examples include ribavirin, favipiravir (also known as T-705 or Avigan), T-705 monophosphate, T-705 diphosphate, T-705 triphosphate, ST-193, and mixtures thereof. The compounds and compositions of the present disclosure are also intended for use with general care provided patients with infections, including one or more of the following: parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate) (such as a combination of Nitazoxanide, Ribavirin and Ivermectin Plus Zinc Supplement), anti-inflammatory agents (such as ibuprofen, fenretinide, baricitinib (e.g., Olumiant®), such as in combination with remdesivir), pain medications, IL-6 inhibitors, such as sarilumab (e.g., Kevzara® sarilumab) and TZLS-501, losartan, tissue plasminogen activators, such as alteplase (e.g., Activase® alteplase), convalescent plasma, complement inhibitor, and medications for other common diseases in the patient population, such anti-malarial agents (including artemether and artesunate- lumefantrine combination therapy), typhoid (including quinolone antibiotics, such as ciprofloxacin, chloroquine, and hydroxychloroquine, macrolide antibiotics, such as azithromycin, doxycycline, cephalosporin antibiotics, such as ceftriaxone, or aminopenicillins, such as ampicillin), or shigellosis, and other agents such as bemcentinib, one or more steroids such as Dexamethasone, Hydrocortisone, and/or Methylprednisolone, fadraciclib (CYC065), seliciclib (CYC202 or R-roscovitine), AT-100 (rhSP-D), NP-120 (Ifenprodil), BXT-25, tocilizumab (e.g., Acterma® tocilizumab), AmnioBoost, pirfenidone (e.g., Esbriet® pirfenidone), rintatolimod (e.g., Ampligen® rintatolimod), Camrelizumab, thymosin, famotidine, vasopressors, anticoagulant, cyclosporine, melatonin, Chinese herbal treatment (e.g., Polygonum cuspidatum), and antibody therapy (e.g., anti-SARS-CoV-2 polyclonal hyperimmune globulin (H-IG) therapy). In certain embodiments, compositions of the present disclosure are combined with a cell therapy regimen, such as one that boosts immune response. In some instances, compositions are combined with sarilumab.

For the avoidance of doubt, in general the present disclosure embraces products, uses, and methods involving the OCS and any single one or combination of more than one of any of the other active agents described herein. For instance, in embodiments the disclosure extends to an OCS for use in a method, as described herein, wherein the method comprises co-administering the OCS with any one or more of any of the other active agents described herein. Co-administering is not limited to simultaneous administration (expressly encompassing also separate, sequential, etc. administration) and can be either of a single medicinal product comprising all active agents or of a plurality of medicinal products comprising separate active agents.

It is also possible to combine the at least one OCS and optional at least one antimicrobial with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of the at least one OCS and optional at least one antimicrobial with one or more other active therapeutic agents generally refers to simultaneous or sequential administration of the at least one OCS and optional at least one antimicrobial and one or more other active therapeutic agents, such that therapeutically effective amounts of the at least one OCS and optional at least one antimicrobial and one or more other active therapeutic agents are both present in the body of the patient.

Co-administration includes administration of unit dosage(s) of the at least one OCS and optional at least one antimicrobial before or after administration of unit dosage(s) of one or more other active therapeutic agents, for example, administration of the at least one OCS and optional at least one antimicrobial within seconds, minutes, or hours of the administration of one or more other active therapeutic agents. For example, unit dose(s) of the at least one OCS and optional at least one antimicrobial can be administered first, followed within seconds or minutes by administration of unit dose(s) of one or more other active therapeutic agents. Alternatively, unit dose(s) of one or more other therapeutic agents can be administered first, followed by administration of unit dose(s) of the at least one OCS and optional at least one antimicrobial within seconds or minutes. In some cases, it may be desirable to administer unit dose(s) of the at least one OCS and optional at least one antimicrobial first, followed, after a period of hours (e.g., 1-12 hours), by administration of unit dose(s) of one or more other active therapeutic agents. In other cases, it may be desirable to administer unit dose(s) of one or more other active therapeutic agents first, followed, after a period of hours (e.g., 1-12 hours), by administration of unit dose of the at least one OCS and optional at least one antimicrobial.

The combination therapy may provide “synergy” and be “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect which is greater than the predicted purely additive effects of the individual compounds of the combination.

In certain embodiments, methods and compositions of the present disclosure are sufficient to reduce the amount of one or more elevated serum liver enzymes. In some instances, the subject methods and compositions are sufficient to reduce serum alanine aminotransferase (ALT), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum ALT by 40% or more. In certain instances, administering 25HC3S is sufficient to reduce the amount of serum ALT to an amount that is below the upper limit of normal levels of ALT.

In certain embodiments, the subject methods and compositions are sufficient to reduce serum aspartate aminotransferase (AST), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum AST by 40% or more. In certain instances, administering 25HC3S is sufficient to reduce the amount of serum AST to an amount that is below the upper limit of normal levels of AST.

In certain embodiments, the subject methods and compositions are sufficient to reduce bilirubin, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum bilirubin by 40% or more. In certain instances, administering 25HC3S is sufficient to reduce the amount of serum bilirubin to an amount that is below the upper limit of normal levels of bilirubin.

In certain embodiments, the subject methods and compositions are sufficient to reduce creatinine, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum creatinine by 40% or more. In certain instances, administering 25HC3 S is sufficient to reduce the amount of serum creatinine to an amount that is below the upper limit of normal levels of creatinine.

In certain embodiments, the subject methods and compositions are sufficient to reduce creatinine kinase, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum creatinine kinase by 40% or more. In certain instances, administering 25HC3S is sufficient to reduce the amount of serum creatinine kinase to an amount that is below the upper limit of normal levels of creatinine kinase.

In certain embodiments, the subject methods and compositions are sufficient to reduce C-reactive protein (CRP), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum CRP by 40% or more. In certain instances, administering 25HC3S is sufficient to reduce the amount of serum CRP to an amount that is below the upper limit of normal levels of CRP.

In certain embodiments, the subject methods and compositions are sufficient to increase forced expiratory volume, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including increasing forced expiratory volume by 40% or more. In certain instances, administering 25HC3S is sufficient to increase forced expiratory volume to an amount that is above the lower limit of normal levels of forced expiratory volume.

In certain embodiments, methods and compositions of the present disclosure are sufficient to reduce the duration of hospitalization. In certain embodiments, the subject methods and compositions are sufficient to reduce the duration of hospitalization, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the duration of hospitalization by 40% or more, as compared to otherwise identical treatment without administration of 25HC3S.

In certain embodiments, methods and compositions of the present disclosure are sufficient to reduce the duration of intensive care unit (ICU) stay. In certain embodiments, the subject methods and compositions are sufficient to reduce the duration of ICU stay, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the duration of ICU stay by 40% or more, as compared to otherwise identical treatment without administration of 25HC3S.

In certain embodiments, methods and compositions of the present disclosure are sufficient to reduce the likelihood of mechanical ventilation. In certain embodiments, the subject methods and compositions are sufficient to reduce the likelihood of mechanical ventilation, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the likelihood of mechanical ventilation by 40% or more, as compared to otherwise identical treatment without administration of 25HC3S.

In certain embodiments, methods and compositions of the present disclosure are sufficient to reduce the severity or occurrence of ascites. In certain embodiments, the subject methods and compositions are sufficient to reduce the severity or occurrence of ascites such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the severity or occurrence of ascites by 40% or more, as compared to otherwise identical treatment without administration of 25HC3S.

In certain embodiments, methods and compositions of the present disclosure are sufficient to reduce the likelihood of organ failure, e.g., liver failure or renal failure. In certain embodiments, the subject methods and compositions are sufficient to reduce the likelihood of organ failure, e.g., liver failure or renal failure, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the likelihood of organ failure, e.g., liver failure or renal failure, by 40% or more, as compared to otherwise identical treatment without administration of 25HC3S.

In certain embodiments, methods and compositions of the present disclosure are sufficient to reduce the likelihood of renal failure. In certain embodiments, the subject methods and compositions are sufficient to reduce the likelihood of organ failure, e.g., liver failure or renal failure, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the likelihood of renal failure, by 40% or more, as compared to otherwise identical treatment without administration of 25HC3S.

In certain embodiments, methods and compositions of the present disclosure are sufficient to reduce the likelihood of liver failure. In certain embodiments, the subject methods and compositions are sufficient to reduce the likelihood of liver failure, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the likelihood of liver failure by 40% or more, as compared to otherwise identical treatment without administration of 25HC3S.

In certain embodiments, methods and compositions of the present disclosure are sufficient to reduce the occurrence or severity of hepatorenal syndrome. In certain embodiments, the subject methods and compositions are sufficient to reduce the occurrence or severity of hepatorenal syndrome, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the occurrence or severity of hepatorenal syndrome by 40% or more, as compared to otherwise identical treatment without administration of 25HC3S.

In certain embodiments, methods and compositions of the present disclosure are sufficient to reduce the likelihood of renal replacement therapy. In certain embodiments, the subject methods and compositions are sufficient to reduce the likelihood of renal replacement therapy, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the likelihood of renal replacement therapy by 40% or more, as compared to otherwise identical treatment without administration of 25HC3S.

In certain embodiments, methods and compositions of the present disclosure are sufficient to reduce the duration of renal replacement therapy. In certain embodiments, the subject methods and compositions are sufficient to reduce the duration of renal replacement, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the duration of renal replacement therapy by 40% or more, as compared to otherwise identical treatment without administration of 25HC3S.

In certain embodiments, methods and compositions of the present disclosure are sufficient to reduce the severity or occurrence of hepatic encephalopathy. In certain embodiments, the subject methods and compositions are sufficient to reduce the severity or occurrence of hepatic encephalopathy, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the severity or occurrence of hepatic encephalopathy by 40% or more, as compared to otherwise identical treatment without administration of 25HC3S.

In certain embodiments, methods and compositions of the present disclosure are sufficient to reduce the severity or occurrence of coagulopathy. In certain embodiments, the subject methods and compositions are sufficient to reduce the severity or occurrence of coagulopathy, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the severity or occurrence of coagulopathy by 40% or more, as compared to otherwise identical treatment without administration of 25HC3S.

In certain embodiments, methods and compositions of the present disclosure are sufficient to increase survival. In certain embodiments, the subject methods and compositions are sufficient to increase survival, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including increasing survival by 40% or more, as compared to otherwise identical treatment without administration of 25HC3S.

The present invention will be further illustrated by way of the following Examples. These Examples are non-limiting and do not restrict the scope of the invention. Unless stated otherwise, all percentages, parts, etc. presented in the Examples are by weight.

### EXAMPLES

### Example 1—Acute Organ Injury in Mice

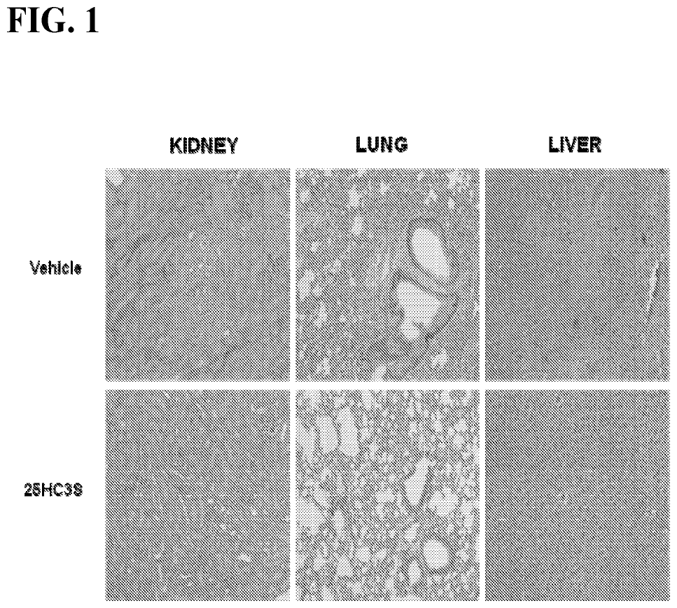

C57BL/6J mice, 21 week old, received an i.p. injection of 25HC3S sodium salt (50 mg/kg) or vehicle. Two hours later, mice were challenged with an intravenous injection of lipopolysaccharide (LPS) at 5 mg/kg. For vehicle-treated mice, the tissues, liver, lung, and kidney, were collected right before when animals met all criteria to be sacrificed. For mice treated with 25HC3S sodium salt, tissues were collected at the day 3, when 80% of vehicle-treated mice died. The tissue sections were stained with hematoxylin and eosin, examined under light microscope with magnification x 100. FIG. 1 shows that treatment with 25HC3 S sodium salt improved the health of kidney, lung, and liver tissues.

### Example 2—Acute Organ Injury in Mice

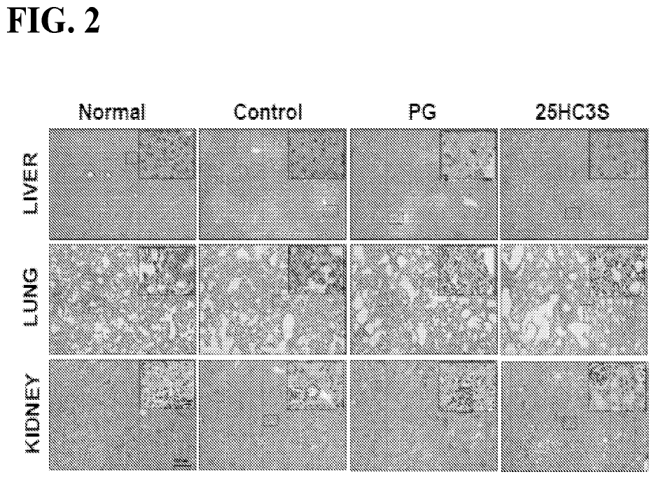

Normal: normal mice without treatment;
Control: mice with 10% glucose and acetaminophen (APAP) injection (600 mg/kg APAP for 24hr);
PG: mice with vehicle and APAP injection (600 mg/kg APAP for 24hr);
PG+25HC3S: mice with 25HC3S (25 mg/kg) and APAP injection.
FIG. 2 shows that treatment with 25HC3S sodium salt improved kidney, lung, and liver tissues.

### Example 3—(Prophetic)

A Randomized, Double-Blind, Placebo Controlled Study to Evaluate Safety of 25HC3S in Subjects Infected with SARS-CoV-2 (e.g., diagnosed as having COVID-19) with Acute or Chronic Liver Disease or Kidney Disease

| Phase of | Phase 2a | |
|---|---|---|
| Development: | | |
| Objectives and | Primary: Evaluate the safety in subjects treated with 25HC3S as | |
| Primary Endpoint: | evidenced by treatment-emergent adverse events (TEAEs) | |
| Trial Design: | This is a Phase 2a, randomized, double-blind, placebo control study | |
| | to evaluate safety. A total of 40 subjects will be enrolled into the | |
| | following 2 dosing groups at 3:1 ratio: | |
| | 25HC3S sodium salt: 150 mg (5 mL of 30 mg/mL) on Day 1 | |
| | and on Day 4, | |
| | Placebo: Sterile Water for Injection (5 mL) on Day 1 and | |
| | on Day 4 | |
| | Patients will be followed up for 28 days | |
| | During the trial, subjects should receive standard of care as | |
| | determined by the PI. | |
| Trial Population: | Subjects diagnosed with SARS-CoV-2 infection (having COVID-19) | |
| | with acute or chronic liver disease or kidney disease. | |
| Inclusion Criteria: | 1. | Able to provide written informed consent (either from subject |
| | | or subject's legally acceptable representative) |
| | 2. | Hospitalized with documented COVID-19 infection with RT |
| | | PCR testing |
| | 3. | Acute or acute-on-chronic liver injury defined as ALT >5x |
| | | upper limit of population reference range and bilirubin >2x |
| | | upper limit of normal (ULN); or |
| | 4. | Acute kidney injury stage 2 by Kidney Disease Improving |
| | | Global Outcomes (KDIGO) criteria (i.e., at least doubling of |
| | | serum creatinine from a known baseline value obtained within |
| | | 12 months); or |
| | 5. | Chronic kidney disease (CKD EPI eGFR between |
| | | 15-50 mL/min/1.73 m²) with acute liver injury (ALT >2x upper |
| | | limit of population reference range) |
| | 6. | Women of child-bearing potential (defined as females who are |
| | | not surgically sterile or who are not over the age of 52 and |
| | | amenorrhoeic for at least 12 months) must utilize appropriate |
| | | birth control throughout the study duration. Acceptable |
| | | methods that may be used are abstinence, birth control pills |
| | | (“The Pill”) or patch, diaphragm, IUD (coil), vaginal ring, |
| | | condom, surgical sterilization or progestin implant or injection, |
| | | or sexual activity limited to a sterile (e.g., vasectomized) male |
| | | partner. |
| | 7. | Male subjects must agree to use a medically acceptable |
| | | method of contraception/birth control and refrain from sperm |
| | | donation throughout the study duration. |

| | |
|---|---|
| Safety Evaluation: | Primary endpoint: |
| | Percentage of Participants with Treatment-Emergent (TE) |
| | Serious AEs (SAE) |
| Safety | The following parameters will be recorded for the safety |
| Assessments: | evaluation: |
| | Treatment-emergent AEs |
| | Vital Signs |
| | Clinical laboratory measurements |
| | Physical Examination |
| Test drug, dosage and | 25HC3S sodium salt at 150 mg (5 mL at 30 mg/mL) diluted in |
| mode of administration: | 100 mL of 5% dextrose or 0.9% sodium chloride and infused |
| | intravenously over approximately 2 hours. |
| Comparator, dosage and | Sterile Water for Injection 5 mL diluted in 100 mL of 5% dextrose or |
| mode of administration: | 0.9% sodium chloride and infused intravenously over approximately |
| | 2 hours. |

### Example 4—(Prophetic)

A Randomized, Double-Blind, Placebo Controlled Study to Evaluate Safety and Efficacy of 25HC3S in Subjects Infected with SARS-CoV-2 with Acute Liver or Kidney Injury

| Phase of | Phase 2 | |
|---|---|---|
| Development: | | |
| Objectives and | Primary: | |
| Primary | Evaluate safety in subjects treated with 25HC3S sodium salt as | |
| Endpoints: | evidenced by treatment-emergent serious adverse events (TESAEs) | |
| | Evaluate efficacy of 25HC3S sodium salt in treatment of acute organ | |
| | injury and/or failure, such as acute liver or kidney injury and/or | |
| | failure, in subjects infected with SARS-CoV-2. The primary efficacy | |
| | endpoint is the composite of survival and being free of acute organ | |
| | failure at Day 28. | |
| Trial Design: | This is a Phase 2, randomized, double-blind, placebo controlled study to | |
| | evaluate safety and efficacy of 25HC3S sodium salt. A total of 80 | |
| | subjects will be enrolled into the following two study treatment groups in | |
| | a 3:1 (25HC3S sodium salt:Placebo) ratio: | |
| | 25HC3S sodium salt: 150 mg (5 mL of 30 mg/mL in 100 mL | |
| | infusion fluid containing 5% dextrose or 0.9% sodium chloride) on | |
| | Day 1 and on Day 4 | |
| | Placebo: Sterile Water for Injection (5 mL in 100 mL infusion fluid | |
| | containing 5% dextrose or 0.9% sodium chloride) on Day 1 and on | |
| | Day 4 | |
| Trial Population: | Subjects diagnosed with SARS-CoV-2 infection with acute liver injury or | |
| | acute kidney injury (AKI) | |
| Inclusion Criteria: | 1. | Age 18-80 years old and able to provide written informed consent |
| | | (either from subject or subject's legally acceptable representative) |
| | 2. | Hospitalized with documented COVID-19 infection diagnosed by |
| | | standard RT-PCR or equivalent testing other than anti-COVID-19 |
| | | antibody testing |
| | 3. | Hospitalized with moderate, severe, or early critical COVID-19 |
| | | illness |
| | 4. | Subject meets one of the following liver or kidney criteria at the time |
| | | of enrollment: |

| | |
|---|---|
| a. | Acute liver injury or acute on chronic liver disease as defined by |
| | ALT > 5x ULN or > 3x baseline. |
| b. | AKI: Stage 2 AKI by Kidney Disease Improving Global |
| | Outcomes (KDIGO) criteria. |

| | |
|---|---|
| 5. | Women of child-bearing potential (defined as women gender |
| | assigned at birth) who are not surgically sterile or who are not over |
| | the age of 52 and amenorrhoeic for at least 12 months) must utilize |
| | appropriate birth control throughout the study duration. Acceptable |
| | methods that may be used are abstinence, birth control pills (“The |
| | Pill”) or patch, diaphragm, IUD (coil), vaginal ring, condom, surgical |
| | sterilization or progestin implant or injection, or sexual activity |
| | limited to a sterile (e.g., vasectomized) male partner. |
| 6. | Male subjects must agree to use a medically acceptable method of |
| | contraception/birth control and refrain from sperm donation |
| | throughout the study duration |

| | |
|---|---|
| Test drug, dosage and | 25HC3S sodium salt at 150 mg (5 mL of 30 mg/mL) diluted in 100 mL |
| mode of administration: | of 5% dextrose or 0.9% sodium chloride and infused intravenously |
| | over approximately 2 hours. |
| Comparator, dosage and | Sterile Water for Injection 5 mL diluted in 100 mL of 5% dextrose or |
| mode of administration: | 0.9% sodium chloride and infused intravenously over approximately |
| | 2 hours. |

1. A method of treating an infectious disease in a human subject in need thereof, the method comprising administering to the subject at least one antimicrobial and at least one oxygenated cholesterol sulfate (OCS) in an amount that is sufficient to treat the infectious disease.

2. The method of claim 1, wherein the at least one antimicrobial comprises at least one member selected from antifungals, antivirals, antiparasitics, and antibacterials.

3. The method of claim 1, wherein the at least one antimicrobial comprises remdesivir.

4. A method of preventing or treating dysfunction or failure of one or more organs or organ systems in a human subject in need thereof and suffering from an infectious disease, comprising:
administering to the subject an amount of at least one oxygenated cholesterol sulfate (OCS) that is sufficient to prevent or treat the dysfunction or failure of the organ or organ system.

5. A method of preventing or treating tissue damage of one or more organs or organ systems in a human subject in need thereof and suffering from an infectious disease, comprising:
administering to the subject an amount of at least one oxygenated cholesterol sulfate (OCS) that is sufficient to prevent or treat tissue damage of the organ or organ system.

6. A method of treating an infectious disease in a human subject in need thereof, the method comprising administering to the subject at least one oxygenated cholesterol sulfate (OCS) in an amount that is sufficient to treat the infectious disease.

7. At least one antimicrobial and at least one OCS for use in a method of treating an infectious disease in a human subject in need thereof, wherein the method is as defined in any one of claims 1 to 6.

8. Use of at least one antimicrobial and at least one OCS thereof in a method for the manufacture of a medicament for use in a method of treating an infectious disease in a human subject in need thereof, wherein the method is as defined in any one of claims 1 to

6.

9. A method comprising administering to a subject diagnosed with COVID-19 a therapeutically effective amount of remdesivir and 25HC3S or a salt thereof.

10. Use of remdesivir and 25HC3S or a salt thereof in the treatment of COVID-19

11. A composition comprising remdesivir and 25HC3S or a salt thereof for use in the treatment of COVID-19.

12. Use of remdesivir and 25HC3S or a salt thereof in the manufacture of a medicament for use in a method of treating COVID-19.

13. An OCS for use in a method for treatment of a human subject, wherein the method is as defined in any one of claims 1 to 6.

14. Use of an OCS in the manufacture of a medicament for use in a method for treatment of a human subject, wherein the method is as defined in any one of claims 1 to 6.

15. An OCS for use in a method for treatment of a human subject by co-administration with an antimicrobial, wherein the OCS, the method and the antimicrobial are as defined in any one of claims 1 to 6.

16. An antimicrobial for use in a method for treatment of a human subject by co- administration with an OCS, wherein the OCS, the method and the antimicrobial are as defined in any one of claims 1 to 6.

17. Use of an OCS in the manufacture of a medicament for use in a method for treatment of a human subject by co-administration with an antimicrobial, wherein the OCS, the method and the antimicrobial are as defined in any one of claims 1 to 6.

18. Use of an antimicrobial in the manufacture of a medicament for use in a method for treatment of a human subject by co-administration with an OCS, wherein the OCS, the method and the antimicrobial are as defined in any one of claims 1 to 6.

19. A product comprising: (a) an OCS; and (b) an antimicrobial; as a combined preparation for simultaneous, concurrent, separate or sequential use in a method for treatment of a human subject, wherein the OCS, the method and the antimicrobial are as defined in any one of claims 1 to 6.
